(19) United States
(12) Patent Application Publication
(10) Pub. No.: US 2015/0094641 A1
(43) Pub. Date: Apr. 2, 2015

(54) ANTIPROLIFERATIVE SURFACE MODIFICATIONS AND METHODS OF USE

(71) Applicant: THE REGENTS OF THE UNIVERSITY OF COLORADO, Denver, CO (US)

(72) Inventors: Daewon Park, Englewood, CO (US); Malik Y. Kahook, Denver, CO (US); Amin Famili, Denver, CO (US)

(21) Appl. No.: 14/396,941

(22) PCT Filed: Apr. 26, 2013
(86) PCT No.: PCT/US13/38360

§ 371 Date: Oct. 24, 2014

Related U.S. Application Data
(60) Provisional application No. 61/640,843, filed on May 1, 2012.

(51) Int. Cl. A61F 9/007

(52) U.S. Cl. CPC A61F 9/00781; A61F 2210/0014
USPC 604/8

(57) ABSTRACT

This invention is in the field of medicinal intervention. The present invention relates to a device constructed from metals, polymers or other materials that are amenable to precise surface modifications and coupling with erodible agents methods for its use, wherein (1) the erodible agents, which contain active ingredients (medications), provide for acute control of cellular proliferation and (2) a pattered surface having milli, micron, and/or nano-sized micro-patterning characteristics imparts anti-proliferative properties over the long-term.

### CROSS-REFERENCE TO RELATED APPLICATIONS

The present application claims the benefit of U.S. Provisional Patent Application No. 61/640,843, filed on. May 1, 2012, which is incorporated herein by reference [1].

### FIELD OF THE INVENTION

This invention is in the field of implantable medical devices. The present invention relates to a device constructed from metals, polymers or other materials that are amenable to precise surface modifications and coupling with erodible agents methods for its use, wherein (1) the erodible agents, which contain active ingredients (i.e., for example, medications) provide for acute control of cellular proliferation and (2) a pattered surface having micron-, and/or nano-sized micro-patterning characteristics that imparts anti-proliferative properties.

### BACKGROUND OF THE INVENTION

A major problem in implanted devices is the proliferation of fibroblasts and other cells on the device surface and the formation of inflammation, scar tissue and encapsulation. The disorganized growth of fibroblasts and the inflammatory response elicited by the presence of the implant alter the function of implants. These responses most often result in formation of a dense, fibrous capsule surrounding the implant. In many applications, this fibrous capsule can negatively impact proper functioning of the implanted device, for example by preventing diffusion of molecules between the implant and its environment or by generally altering the local physiological environment. As wound modulation has both an acute and chronic phase, it is important to address both phases to result in optimal medical and surgical outcomes. What is needed is an implant that would reduce or mitigate the proliferation of cells through both primary (acute and short term) and secondary means (chronic/long-term).

### SUMMARY OF THE INVENTION

This invention is in the field of implantable medical devices. The present invention relates to a device constructed from metals, polymers or other materials that are amenable to precise surface modifications and coupling with erodible agents methods for its use, wherein (1) the erodible agents, which contain active ingredients (i.e., for example, medications) provide for acute control of cellular proliferation and (2) a pattered surface having micron-, and/or nano-sized micro-patterning characteristics that imparts anti-proliferative properties.

In one embodiment, the invention relates to a device comprising an anti-proliferative surface, wherein said surface comprises a micro-patterned geometrical pattern, said pattern having a plurality of grooves between a plurality of raised surfaces. In one embodiment, said pattern is selected from the group consisting of vertical, horizontal, circular, intersecting grid, and concentric rings. In one embodiment, said grooves comprise a plurality of medication depots such that the top of said depots are below said plurality of raised surfaces. In one embodiment, said plurality of raised surfaces are separated by a distance of approximately 10-50 μm. In one embodiment, said plurality of raised surfaces are separated by a distance of approximately 20-35 μm. In one embodiment, said plurality of raised surfaces are separated by a distance of approximately 20-25 μm. In one embodiment, said grooves are at least as deep as the distance separating said plurality of raised surfaces. In one embodiment, the depths of said grooves are deeper than the distance separating said plurality of raised surfaces wherein said grooves comprise a plurality of medication depots. In one embodiment, said medication depots are at least 25 μm below said raised surfaces. In one embodiment, said medication depot comprises an anti-proliferative material. In one embodiment, said geometrical pattern inhibits cellular proliferation, cell attachment, cell migration or release of specific factors. In one embodiment, said device is an implanted medical device. In one embodiment, said implanted medical device is in an ocular region. In one embodiment, said ocular region is selected from the group consisting of the sclera, Schlemm's canal and the suprachoroidal space. In one embodiment, said surface further comprises silicone, polyimide (PI), polysulfone (PES), polyetheretherketone (PEEK), polypropylene, polyetherimide (PEI), titanium, nitinol, stainless steel, gold, hydrophilic or hydrophopic polymers, shape memory polymers or alloys, ceramics, alloys, silicates, or other materials. In one embodiment, said device has shape selected from the group consisting of spherical, non-spherical (egg-shaped), cylindrical, rectangular, cubic, toroidal, conical, cuboidal, pyramidal, prism, and planar shapes. In one embodiment, said device has a cylindrical shape. In one embodiment, said device contains at least one lumen. In one embodiment, said lumen contains a depot. In one embodiment, said medication depot contains at least one medication. In one embodiment, said medication is selected from the group comprising anti-fibrotic agent, anti-inflammatory agent, immunosuppressant agent, anti-neoplastic agent, migration inhibitors, anti-proliferative agent, rapamycin, triamcinolone acetonide, everolimus, tacrolimus, paclitaxel, actinomycin, azathioprine, dexamethasone, cyclosporine, bevacizumab, an anti-VEGF agent, an anti-IL-1 agent, canakinumab, an anti-IL-2 agent, viral vectors, beta blockers, alpha agonists, muscarinic agents, steroids, antibiotics, non-steroidal anti-inflammatory agents, prostaglandin analogues, ROCK inhibitors, nitric oxide, endothelin, matrixmetalloproteinase inhibitors, CNPA, corticosteroids, and/or antibody-based immunosuppresants. In one embodiment, said medication is combined with a polymer. In one embodiment, wherein said polymer is selected from the group comprising poly(lactic-co-glycolic acid), polyethylene glycol, poly(lactic acid), poly(glycolic acid), poly(amido ester), polyethylene terephthalate, poly(caprolactone), poly(hydroxy butyrate), poly(butylene succinate), poly(vinyl alcohol), poly(hydroxybutyrate), poly(methyl acrylate), poly(methyl methylmethacrylate), poly(sebacic acid), carboxymethyl cellulose, ethyl cellulose, cellulose acetate, polydioxanone, or polymers from the categories: polyesters, polyanhydrides, polyamides, polycyanoacrylates, polyurethanes, polyorthoesters, silicones, acrylic polymers, cellulose derivatives and/or poloxamers. In one embodiment, said grooves are patterned in a vertical orientation. In one embodiment, said grooves are patterned in a horizontal orientation. In one embodiment, said grooves are patterned in a diagonal orientation. In one embodiment, said grooves are patterned in a helical orientation. In one embodiment, said geometrical pattern further comprises a columnar structure. In one embodiment, said device is a catheter. In one embodiment, said device is a stent. In one embodiment, said catheter comprises a defibrillation device. In one embodiment, said device is an intravenous catheter. In one embodiment, said device is a Hickman catheter. In one embodiment, said device is a mesh prosthesis. In one embodiment, said device is a hernia mesh. In one embodiment, said device is a Baerveldt glaucoma implant. In one embodiment, said device is a dental implant. In one embodiment, said device is a glaucoma shunting device. In one embodiment, said geometrical pattern prevents encapsulation. In one embodiment, said geometrical pattern prevents disorderly growth of fibroblasts. In one embodiment, said geometrical pattern prevents the formation of scar tissue. In one embodiment, said geometrical pattern prevents cellular proliferation. In one embodiment, said geometrical pattern inhibits cellular attachment. In one embodiment, said geometrical pattern provides fluid drainage.

In one embodiment, the invention relates to a method of treating a subject in need of inhibiting cellular proliferation comprising: a) providing a drug delivery device comprising an anti-proliferative surface, wherein said surface comprises a micro-patterned geometrical pattern, said pattern having a plurality of grooves between a plurality of raised surfaces, wherein said grooves comprise a plurality of medication depots such that the top of said depots are below said plurality of raised surfaces; and b) delivering a medication from said medication depot to inhibit cellular proliferation. In one embodiment, said pattern is selected from the group consisting of vertical, horizontal, circular, intersecting grid, and concentric rings. In one embodiment, said plurality of raised surfaces are separated by a distance of approximately 10-50 μm. In one embodiment, said plurality of raised surfaces are separated by a distance of approximately 20-35 μm. In one embodiment, said plurality of raised surfaces are separated by a distance of approximately 20-25 μm. In one embodiment, said grooves are at least as deep as the distance separating said plurality of raised surfaces. In one embodiment, the depths of said grooves are deeper than the distance separating said plurality of raised surfaces wherein said grooves comprise a plurality of medication depots. In one embodiment, said medication depots are at least 25 μm below said raised surfaces. In one embodiment, said wherein said medication comprises an anti-proliferative material. In one embodiment, said geometric pattern inhibits cellular proliferation, cell attachment, cell migration or release of specific factors. In one embodiment, said device is an implanted medical. In one embodiment, said implanted medical device is in an ocular region. In one embodiment, said ocular region is selected from the group comprising the sclera, Schlemm's canal and the suprachoroidal space. In one embodiment, said device further comprises silicone, polyimide (PI), polysulfone (PES), polyetheretherketone (PEEK), polypropylene, polyetherimide (PEI), titanium, nitinol, stainless steel, gold, hydrophilic or hydrophopic polymers, shape memory polymers, ceramics, alloys, silicates, or other materials. In one embodiment, said device has shape selected from the group consisting of spherical, non-spherical (egg-shaped), cylindrical, rectangular, cubic, toroidal, conical, cuboidal, pyramidal, prism, and planar shapes. In one embodiment, said device has a cylindrical shape. In one embodiment, said device contains at least one lumen. In one embodiment, said lumen contains a depot. In one embodiment, said medication is selected from the group comprising anti-fibrotic agent, anti-inflammatory agent, immunosuppressant agent, anti-neoplastic agent, migration inhibitors, anti-proliferative agent, rapamycin, triamcinolone acetonide, everolimus, tacrolimus, paclitaxel, actinomycin, azathioprine, dexamethasone, cyclosporine, bevacizumab, an anti-VEGF agent, an anti-IL-1 agent, canakinumab, an anti-IL-2 agent, viral vectors, beta blockers, alpha agonists, muscarinic agents, steroids, antibiotics, non-steroidal anti-inflammatory agents, prostaglandin analogues, ROCK inhibitors, nitric oxide, endothelin, matrixmetalloproteinase inhibitors, CNPA, corticosteroids, and antibody-based immunosuppresants. In one embodiment, said medication is combined with a polymer. In one embodiment, said polymer is selected from the group comprising poly(lactic-co-glycolic acid), polyethylene glycol, poly(lactic acid), poly(glycolic acid), poly(amido ester), polyethylene terephthalate, poly(caprolactone), poly(hydroxy butyrate), poly(butylene succinate), poly(vinyl alcohol), poly(hydroxybutyrate), poly(methyl acrylate), poly(methyl methylmethacrylate), poly(sebacic acid), carboxymethyl cellulose, ethyl cellulose, cellulose acetate, polydioxanone, or polymers from the categories: polyesters, polyanhydrides, polyamides, polycyanoacrylates, polyurethanes, polyorthoesters, silicones, acrylic polymers, cellulose derivatives and/or poloxamers. In one embodiment, said grooves are patterned in a vertical orientation. In one embodiment, said grooves are patterned in a horizontal orientation. In one embodiment, said grooves are patterned in a diagonal orientation. In one embodiment, said grooves are patterned in a helical orientation. In one embodiment, said geometrical pattern further comprises a columnar structure. In one embodiment, said device is a catheter. In one embodiment, said device is a stent. In one embodiment, said device is a catheter for a defibrillation device. In one embodiment, said device is an intravenous catheter. In one embodiment, said device is a Hickman catheter. In one embodiment, said device is a mesh prosthesis. In one embodiment, said device is a hernia mesh. In one embodiment, said device is a Baerveldt glaucoma implant. In one embodiment, said device is a dental implant. In one embodiment, said device is a glaucoma aqueous shunting device. In one embodiment, said device is a device that shunts fluid from one area to another. In one embodiment, said geometrical pattern prevents encapsulation. In one embodiment, said geometrical pattern prevents disorderly growth of fibroblasts. In one embodiment, said geometrical pattern prevents the formation of scar tissue. In one embodiment, said geometrical pattern prevents cellular proliferation. In one embodiment, said geometrical pattern inhibits cellular attachment. In one embodiment, said geometrical pattern provides fluid drainage.

In one embodiment, the invention relates to a method of treating a subject in need of inhibiting cellular proliferation comprising: a) providing an implanted device comprising an anti-proliferative surface, wherein said surface comprises a micro-patterned geometrical pattern, said pattern having a plurality of grooves between a plurality of raised surfaces; and b) using said device to inhibit cellular proliferation. In one embodiment, said pattern is selected from the group consisting of vertical, horizontal, circular, intersecting grid, and concentric rings. In one embodiment, said plurality of raised surfaces are separated by a distance of approximately 10-50 μm. In one embodiment, said plurality of raised surfaces are separated by a distance of approximately 20-35 μm. In one embodiment, said plurality of raised surfaces are separated by a distance of approximately 20-25 μm. In one embodiment, said grooves are at least as deep as the distance separating said plurality of raised surfaces. In one embodiment, said geometric pattern inhibits cellular proliferation, cell attachment, cell migration or release of specific factors. In one embodiment, said device is an implanted medical device. In one embodiment, said implanted medical device is in an ocular region. In one embodiment, said ocular region is selected from the group consisting of the sclera, Schlemm's canal and the suprachoroidal space. In one embodiment, said device further comprises silicone, polyimide (PI), polysulfone (PES), polyetheretherketone (PEEK), polypropylene, polyetherimide (PEI), titanium, nitinol, stainless steel, gold, hydrophilic or hydrophopic polymers, shape memory polymers, ceramics, alloys, silicates, or other materials. In one embodiment, device has shape selected from the group consisting of spherical, non-spherical (egg-shaped), cylindrical, rectangular, cubic, toroidal, conical, cuboidal, pyramidal, prism, and planar shapes. In one embodiment, said device has a cylindrical shape. In one embodiment, said device contains at least one lumen. In one embodiment, said grooves are patterned in a vertical orientation. In one embodiment, said grooves are patterned in a horizontal orientation. In one embodiment, said grooves are patterned in a diagonal orientation. In one embodiment, said grooves are patterned in a helical orientation. In one embodiment, said geometrical pattern further comprises a columnar structure. In one embodiment, said device is a catheter. In one embodiment, said device is a stent. In one embodiment, said device is a catheter for a defibrillation device. In one embodiment, said device is an intravenous catheter. In one embodiment, said device is a Hickman catheter. In one embodiment, said device is a mesh prosthesis. In one embodiment, said device is a hernia mesh. In one embodiment, said device is a Baerveldt glaucoma implant. In one embodiment, said device is a dental implant. In one embodiment, said device is a glaucoma aqueous shunting device. In one embodiment, said device is a device that shunts fluid from one area to another. In one embodiment, said geometrical pattern prevents encapsulation. In one embodiment, said geometrical pattern prevents disorderly growth of fibroblasts. In one embodiment, said geometrical pattern prevents the formation of scar tissue. In one embodiment, said geometrical pattern prevents cellular proliferation. In one embodiment, said geometrical pattern inhibits cellular attachment. In one embodiment, said geometrical pattern provides fluid drainage.

It is not intended that embodiments of the invention be limited to any particular method, medical target, or device confirmation; however, it is believed that the device may be optimally designed to inhibit the proliferation of fibroblasts, smooth muscle cells and other cells on the surface of the implant in both the acute and chronic phases of wound modulation.

### DEFINITIONS

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

As used herein, the terms “medication” or “therapeutic agent” refer to something that treats or prevents or alleviates the symptoms of disease or condition, a drug or pharmaceutical composition. Medication is considered to be delivered or present in therapeutically effective amounts or pharmaceutically effective amounts.

The present invention contemplates the above-described compositions in “therapeutically effective amounts” or “pharmaceutically effective amounts”, which means that amount which, when administered to a subject or patient for treating a disease, is sufficient to effect such treatment for the disease or to ameliorate one or more symptoms of a disease or condition (e.g. ameliorate pain).

As used herein, the terms “treat” and “treating” are not limited to the case where the subject (e.g. patient) is cured and the disease is eradicated. Rather, the present invention also contemplates treatment that merely reduces symptoms, improves (to some degree) and/or delays disease progression. It is not intended that the present invention be limited to instances wherein a disease or affliction is cured. It is sufficient that symptoms are reduced.

As used herein, the terms “medical device,” “implant,” “device,” “medical device,” “medical implant,” “implant/device,” and the like are used synonymously to refer to any object that is designed to be placed partially or wholly within a patient's body for one or more therapeutic or prophylactic purposes such as for tissue augmentation, contouring, restoring physiological function, repairing or restoring tissues damaged by disease or trauma, and/or delivering therapeutic agents to normal, damaged or diseased organs and tissues. While medical devices are normally composed of biologically compatible synthetic materials (e.g., medical-grade stainless steel, titanium and other metals; exogenous polymers, such as polyurethane, silicon, PLA, PLGA, PGA, PCL), other materials may also be used in the construction of the medical implant. While not limiting the present invention to any particular device, specific medical devices and implants that are particularly relevant to this invention include stents, catheters, implanted defribrillators, defribillator leads, cardiac, cerebral, lumbar-peritoneal, peritoneovenous, pulmonary, ocular or other shunts, drug delivery systems, implanted electronic devices, and implanted, microelectromechanical (MEMS) devices. Other devices contemplated include dental implants, hernia mesh devices, encircling bands (beriatric surgery and scleral buckles) and any implant that might be placed in or around the body.

As used herein, the term “medication depot” refers to medication deposited on the bottom level of a micro-patterned geometric pattern, such as a groove.

As used herein, the term “anti-proliferative” refers to refer to agents used or tending to inhibit cell growth.

As used herein, the terms “fibrosis” or “scarring” refers to the formation of fibrous (scar) tissue in response to injury or medical intervention. Therapeutic agents which inhibit fibrosis or scarring can do so through one or more mechanisms including inhibiting inflammation, inhibiting angiogenesis, inhibiting migration or proliferation of connective tissue cells (such as fibroblasts, smooth muscle cells, vascular smooth muscle cells), reducing extracellular matrix (ECM) production or encouraging ECM breakdown, arresting and/or inhibiting cell cycle progression, arresting and/or inhibiting DNA synthesis, and/or inhibiting tissue remodeling. In addition, numerous therapeutic agents described in this invention will have the additional benefit of also reducing tissue regeneration (the replacement of injured cells by cells of the same type) when appropriate.

As used herein, the terms “inhibit fibrosis,” “inhibit scar,” “reduce fibrosis,” “reduce scar,” “fibrosis-inhibitor,” “anti-scarring” and the like are used synonymously to refer to the action of agents or compositions which result in a statistically significant decrease in the formation, deposition and/or maturation of fibrous tissue that may be expected to occur in the absence of the agent or composition.

As used herein, the term “antifibrotic agent” refers to chemical compounds which have antifibrotic activity in mammals. This takes into account the abnormal formation of fibrous connective tissue, which is typically comprised of collagen to a greater or lesser degree. These compounds may have different mechanisms of action, some reducing the formation of collagen or another protein, others enhancing the metabolism or removal of collagen in the affected area of the body. All such compounds having activity in the reduction of the presence of fibrous tissue are included herein, without regard to the particular mechanism of action by which each such drug functions.

As used herein, the terms “encapsulation” as used herein refers to the formation of a fibrous connective tissue capsule (containing fibroblasts, myofibroblasts, inflammatory cells, relatively few blood vessels and a collagenous extracellular matrix) encloses and isolates an implanted prosthesis or biomaterial from the surrounding body tissue. This fibrous tissue capsule, which is the result of unwanted scarring and inflammation in response to an implanted prosthesis or biomaterial, has a tendency to progressively contract, thereby tightening around the implant/biomaterial and causing it to become very firm and disfigured. Further implications of encapsulation and associated contracture include tenderness of the tissue, pain, erosion of the adjacent tissue as well as other complications.

As used herein, the terms “contracture” as used herein refers to permanent or non-permanent scar tissue formation in response to an implanted prosthesis or biomaterial. In general, the condition of contracture involves a fibrotic response that may involve inflammatory components, both acute and chronic. Unwanted scarring in response to an implanted prosthesis or biomaterial can form a fibrous tissue capsule around the area or implantable prosthesis or biomaterial that encloses and isolates it from the surrounding body tissue (as described for encapsulation). Contracture occurs when fibrous tissue capsule matures and starts to shrink (contract) forming a tight, hard capsule around the implant/biomaterial that can alter the anatomy, texture, shape and movement of the implant. In some cases, contracture also draws the overlying skin in towards the implant and leads to dimpling of the skin and disfiguration. Contracture and chronic inflammation can also contribute to tenderness around the implant, pain, and erosion of the adjacent tissue. Fibrotic contractures related to implantation of soft tissue implant/biomaterials may be caused by a variety of factors including surgical trauma and complications, revisions or repeat procedures (the incidence is higher if implantation is being attempted where contractures have occurred previously), inadequate hemostasis (bleeding control) during surgery, aggressive healing processes, underlying or pre-existent conditions, genetic factors (people prone to hypertrohic scar or keloid formation), and immobilization.

As used herein, the terms “implanted” refers to having completely or partially placed a device within a host. A device is partially implanted when some of the device reaches, or extends to the outside of, a host.

As used herein, the term “erodible agent” refers to materials such as polymer or semi-solid gel or the like which are eroded by physiological or chemical processes such that the mass of said agents decreases over the course of implantation. The erodible agent, can be made out of PLGA, Polymers, erodible gels and other materials capable of carrying or containing medications and eroding over time.

As used herein, the term “micro-patterning” preferably refers to millimeter, micrometer, and/or nanometer scale surface modifications including but not limited to laser etching, chemical etching, photo-etching, photolithography, machining, stamping, deposition processes, mechanical drilling, molding, 3D printing, Atomic Layer Deposition or other means of modifying surfaces.

As used herein, the term “anti-inflammatory agent” refers to substance or treatment that reduces inflammation.

As used herein, the term “immunosuppressant agents” refers to drugs that inhibit or prevent activity of the immune system.

As used herein, the term “anti-neoplastic agents” refers to drugs that prevent or inhibit the development, maturation, or spread of neoplastic cells.

As used herein, the term “migration inhibitors” refers to agents that alter the movement of cells in a given environment or that inhibit the migration of specific cell types or cells generally.

As used herein, the term “butylated hydroxy toluene” (abbreviated BHT) refers to a lipophilic (fat-soluble) organic compound, chemically a derivative of phenol, that is useful for its antioxidant properties. BHT is also known as 2,6-bis(1,1-dimethylethyl)-4-methylphenol, 2,6-di-tert-butyl-4-methylphenol, 2,6-di-tert-butyl-p-cresol (DBPC), and 3,5-di-tert-butyl-4-hydroxytoluene. Butylated hydroxy toluene has the structure:

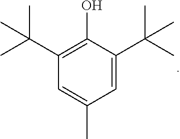 [SMILES: Cc1cc(C(C)(C)C)c(O)c(C(C)(C)C)c1]

As used herein, the term “rapamycin” refers to an immunosuppressant drug used to prevent rejection in organ transplantation.

As used herein, the term “triamcinolone acetonide” refers to a synthetic corticosteroid.

As used herein, the term “everolimus” refers to an immunosuppressant to prevent rejection of organ transplants and treatment of renal cell cancer.

As used herein, the term “tacrolimus” (also FK-506 or fujimycin, trade names Prograf, Advagraf, Protopic) refers to an immunosuppressive drug that is mainly used after allogeneic organ transplant to reduce the activity of the patient's immune system and so lower the risk of organ rejection. It is also used in a topical preparation in the treatment of atopic dermatitis (eczema), severe refractory uveitis after bone marrow transplants, exacerbations of minimal change disease, and the skin condition vitiligo.

As used herein, the term “paclitaxel” refers to a mitotic inhibitor used in cancer chemotherapy.

As used herein, the term “actinomycin” refers to a class of polypeptide antibiotics isolated from soil bacteria of the genus Streptomyces, of which the most significant is actinomycin D.

As used herein, the term “azathioprine” refers to a purine analogue immunosuppressive drug. It is used to prevent rejection following organ transplantation, and to treat a vast array of autoimmune diseases, including rheumatoid arthritis, pemphigus, inflammatory bowel disease (such as Crohn's disease and ulcerative colitis), multiple sclerosis, autoimmune hepatitis, atopic dermatitis, myasthenia gravis, neuromyelitis optica or Devic's disease, restrictive lung disease, and others.

As used herein, the term “dexamethasone” refers to a potent synthetic member of the glucocorticoid class of steroid drugs. It acts as an anti-inflammatory and immunosuppressant.

As used herein, the term “cyclosporine” refers to an immunosuppressant drug widely used in organ transplantation to prevent rejection.

As used herein, the term “bevacizumab” refers to a drug that blocks angiogenesis, the growth of new blood vessels.

As used herein, the term “anti-VEGF agent” refers to a drug that inhibits the action of vascular endothelial growth factor (VEGF).

As used herein, the term “anti-IL-1 agent” refers to a drug that inhibits the action of Interleukin 1 protein.

As used herein, the term “canakinumab” refers to a human monoclonal antibody targeted at interleukin-1 beta.

As used herein, the term “anti-IL-2 agent” refers to a drug that inhibits the action of Interleukin 2 protein.

As used herein, the term “viral vectors” refers to a tool commonly used by molecular biologists to deliver genetic material into cells. A viral vector is modified in such a way as to minimize the risk of handling them. This usually involves the deletion of a part of the viral genome critical for viral replication. Such a virus can efficiently infect cells but, once the infection has taken place, requires a helper virus to provide the missing proteins for production of new virions.

As used herein, the term “beta blockers” (beta-adrenergic blocking agents, beta-adrenergic antagonists, beta-adrenoreceptor antagonists or beta antagonists) refer to a class of drugs used for various indications. They are particularly for the management of cardiac arrhythmias, cardioprotection after myocardial infarction [2] (heart attack), and hypertension [3]. As beta adrenergic receptor antagonists, they diminish the effects of epinephrine (adrenaline) and other stress hormones.

As used herein, the term “alpha agonists” or “α-adrenergic-antagonists” refers to pharmacological agents that act as receptor antagonists of α-adrenergic receptors (α-adrenoceptors).

As used herein, the term “muscarinic agents” refers to a muscarinic receptor agonist or an agent that enhances the activity of the muscarinic acetylcholine receptor.

As used herein, the term “steroids” refers to a type of organic compound that contains a characteristic arrangement of four cycloalkane rings that are joined to each other. Examples of steroids include, but are not limited to, the dietary fat cholesterol, the sex hormones estradiol and testosterone, and the anti-inflammatory drug dexamethasone.

As used herein, the term “antibiotics” refers to a compound or substance that kills or slows down the growth of bacteria, fungus, or other microorganism.

As used herein, the term “non-steroidal anti-inflammatory agents,” “nonsteroidal anti-inflammatory drugs,” usually abbreviated to NSAIDs or NAIDs, but also referred to as nonsteroidal anti-inflammatory agents/analgesics (NSAIAs) or nonsteroidal Anti-inflammatory medicines (NSAIMs), refers to drugs with analgesic and antipyretic (fever-reducing) effects and which have, in higher doses, anti-inflammatory effects.

As used herein, the term “prostaglandin analogues” refers to molecules that are made to bind to a prostaglandin receptor.

As used herein, the term “ROCK inhibitors” refers to a drug that inhibits the action of the rho-associated protein kinase (ROCK).

As used herein, the term “nitric oxide” also known as “nitrogen monoxide” refers to a binary diatomic molecule with chemical formula NO.

As used herein, the term “endothelin” refers to proteins that constrict blood vessels, raise blood pressure, in other embodiments, decrease eye pressure, and protect neuronal tissues from degeneration.

As used herein, the term “matrixmetalloproteinase i” (MMPs) refers to zinc-dependent endopeptidases (capable of degrading all kinds of extracellular matrix proteins, but also can process a number of bioactive molecules); other family members are adamalysins, serralysins, and astacins. The MMPs belong to a larger family of proteases known as the metzincin superfamily. MMPs are also thought to play a major role on cell behaviors such as cell proliferation, migration (adhesion/dispersion), differentiation, angiogenesis, apoptosis, and host defense.

As used herein, the term “matrixmetalloproteinase inhibitors” (MMPs) refers to drugs that inhibit zinc-dependent endopeptidases; other family members are adamalysins, serralysins, and astacins.

As used herein, the term CNP refers to C-Type Natriuretic Peptide.

As used herein, the term “corticosteroids” refers to a class of chemicals that includes steroid hormones naturally produced in the adrenal cortex of vertebrates and analogues of these hormones that are synthesized in laboratories. Corticosteroids are involved in a wide range of physiologic processes, including stress response, immune response, and regulation of inflammation, carbohydrate metabolism, protein catabolism, blood electrolyte levels, and behavior.

As used herein, the term “antibody-based immunosuppresants” refers to immunosuppressant agents that are anti-body based.

As used herein, the term “release of an agent” refers to a statistically significant presence of the agent, or a subcomponent thereof, which has disassociated from the implant and/or remains active on the surface of (or within) the device/implant.

As used herein, the terms “analogue or analog” refer to a chemical compound that is structurally similar to a parent compound but differs slightly in composition (e.g., one atom or functional group is different, added, or removed). An analogue may or may not have different chemical or physical properties than the original compound and may or may not have improved biological and/or chemical activity. For example, the analogue may be more hydrophilic, or it may have altered reactivity as compared to the parent compound. The analogue may mimic the chemical and/or biological activity of the parent compound (i.e., it may have similar or identical activity), or, in some cases, may have increased or decreased activity. The analogue may be a naturally or non-naturally occurring (e.g., recombinant) variant of the original compound. An example of an analogue is a mutein (i.e., a protein analogue in which at least one amino acid is deleted, added, or substituted with another amino acid). Other types of analogues include isomers (enantiomers, diasteromers, and the like) and other types of chiral variants of a compound, as well as structural isomers. The analogue may be a branched or cyclic variant of a linear compound. For example, a linear compound may have an analogue that is branched or otherwise substituted to impart certain desirable properties (e.g., improve hydrophilicity or bioavailability).

As used herein, the term “derivative” refers to a chemically or biologically modified version of a chemical compound that is structurally similar to a parent compound and (actually or theoretically) derivable from that parent compound. A “derivative” differs from an “analogue” in that a parent compound may be the starting material to generate a “derivative,” whereas the parent compound may not necessarily be used as the starting material to generate an “analogue.” An analogue may have different chemical or physical properties of the parent compound. For example, the derivative may be more hydrophilic or it may have altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). The tem′ “derivative” also includes conjugates, and prodrugs of a parent compound (i.e., chemically modified derivatives which can be converted into the original compound under physiological conditions). For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). More detailed information relating to prodrugs is found, for example, in Fleisher et al., Advanced Drug Delivery Reviews 19 (1996) 115 [4] incorporated herein by reference. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups, for example carboxylic acid groups, can form, for example, alkali metal salts or alkaline earth metal salts (e.g., sodium salts, potassium salts, magnesium salts and calcium salts, and also salts with physiologically tolerable quaternary ammonium ions and acid addition salts with ammonia and physiologically tolerable organic amines such as, for example, triethylamine, ethanolamine or tris-(2-hydroxyethyl)amine). Basic groups can form acid addition salts, for example with inorganic acids such as hydrochloric acid, sulfuric acid or phosphoric acid, or with organic carboxylic acids and sulfonic acids such as acetic acid, citric acid, benzoic acid, maleic acid, fumaric acid, tartaric acid, methanesulfonic acid or p-toluenesulfonic acid. Compounds that simultaneously contain a basic group and an acidic group, for example a carboxyl group in addition to basic nitrogen atoms, can be present as zwitterions. Salts can be obtained by customary methods known to those skilled in the art, for example by combining a compound with an inorganic or organic acid or base in a solvent or diluent, or from other salts by cation exchange or anion exchange.

As used herein, the term “inhibitor” refers to an agent that prevents a biological process from occurring or slows the rate or degree of occurrence of a biological process. The process may be a general one such as scarring or refer to a specific biological action such as, for example, a molecular process resulting in release of a cytokine.

As used herein, the term “antagonist” refers to an agent that prevents a biological process from occurring or slows the rate or degree of occurrence of a biological process. While the process may be a general one, typically this refers to a drug mechanism by which the drug competes with a molecule for an active molecular site or prevents a molecule from interacting with the molecular site. In these situations, the effect is that the molecular process is inhibited.

As used herein, the term “agonist” refers to an agent that stimulates a biological process or rate or degree of occurrence of a biological process. The process may be a general one such as scarring or refer to a specific biological action such as, for example, a molecular process resulting in release of a cytokine.

As used herein, the term “anti-microtubule agent” should be understood to include any protein, peptide, chemical, or other molecule that impairs the function of microtubules, for example, through the prevention or stabilization of polymerization. Compounds that stabilize polymerization of microtubules are referred to herein as “microtubule stabilizing agents.” A wide variety of methods may be utilized to determine the anti-microtubule activity of a particular compound, including for example, assays described by Smith et al. (Cancer Lett. 79(2):213-219, 1994) [5] and Mooberry et al., (Cancer Lett. 96(2):261-266, 1995) [6] both incorporated herein by reference.

Any concentration ranges, percentage range, or ratio range recited herein are to be understood to include concentrations, percentages or ratios of any integer within that range and fractions thereof, such as one tenth and one hundredth of an integer, unless otherwise indicated. Also, any number range recited herein relating to any physical feature, such as polymer subunits, size or thickness, are to be understood to include any integer within the recited range, unless otherwise indicated. It should be understood that the terms “a” and “an” as used above and elsewhere herein refer to “one or more” of the enumerated components. For example, “a” polymer refers to both one polymer or a mixture comprising two or more polymers. As used herein, the term “about” means ±15%.

As discussed above, the present invention provides compositions, methods and devices relating to medical and reconstructive devices and implants, which greatly increase their ability to inhibit the formation of reactive scar tissue on, or around, the surface of the implant. In one aspect, the present invention provides for the combination of an anti-scarring agent and a soft tissue implant for use in medical intervention, continuing medical therapy, and/or cosmetic or reconstructive surgery. In one aspect, the present invention is an antifibrotic device for use in medical intervention, continuing medical therapy, and/or cosmetic or reconstructive surgery. In yet another aspect, soft tissue implants are provided that can reduce the development of surrounding scar capsules that harden and contract (also referred to herein as capsular or fibrous contracture), discomfort, leakage of fluid from the implant, infection, asymmetry, and patient dissatisfaction. Described in more detail below are methods for constructing soft tissue implants, compositions and methods for generating medical implants that inhibit fibrosis, and methods for utilizing such medical implants.

As used herein, the term “sclera”, also known as the white of the eye, refers to the opaque, fibrous, protective, outer layer of the eye containing collagen and elastic fiber.

As used herein, the term “stent” refers to an artificial ‘tube’ inserted into a natural passage/conduit in the body to prevent, or counteract, a disease-induced, localized flow constriction. The term may also refer to a tube used to temporarily hold such a natural conduit open to allow access for surgery.

As used herein, the term “shunt” refers to an artificial ‘tube’ inserted into the body to create a hole or passage to allow movement of fluids between two areas. Said tube may be implanted temporarily or may be permanent.

As used herein, the term “catheter” refers to a tube that can be inserted into a body cavity, duct, or vessel. Catheters thereby allow drainage, administration of fluids or gases, or access by surgical instruments. The process of inserting a catheter is catheterization. In most uses, a catheter is a thin, flexible tube (“soft” catheter), though in some uses, it is a larger, solid (“hard”) catheter. A catheter left inside the body, either temporarily or permanently, may be referred to as an indwelling catheter. A permanently inserted catheter may be referred to as a permcath.

As used herein, the term “glaucoma valve” refers to a medical shunt used in the treatment of glaucoma to reduce the eye's intraocular pressure (IOP). There are also several different glaucoma drainage implants. These include the original Molteno implant (1966), the Baerveldt tube shunt, or the valved implants, such as the Ahmed glaucoma valve implant and the later generation pressure ridge Molteno implants. These are indicated for glaucoma patients not responding to maximal medical therapy, with previous failed guarded filtering surgery (trabeculectomy). The flow tube is inserted into the anterior chamber of the eye and the plate is implanted underneath the conjunctiva to allow flow of aqueous fluid out of the eye into a chamber called a bleb.

As used herein, the term “Hickman line” refers to an intravenous catheter most often used for the administration of chemotherapy or other medications, as well as for the withdrawal of blood for analysis. Some types of Hickman lines are used mainly for the purpose of apheresis or dialysis. Hickman lines may remain in place for extended periods and are used when long-term intravenous access is needed.

As used herein, the term “PLGA or poly(lactic-co-glycolic acid)” refers to a copolymer which is used in a host of Food and Drug Administration (FDA) approved therapeutic devices, owing to its biodegradability and biocompatibility. PLGA has been studied for slow drug release [7].

As used herein, the term “polyethylene glycol” (abbreviated PEG) refers to is a polyether compound with many applications in medicine. It has also been known as polyethylene oxide (PEO) or polyoxyethylene (POE), depending on its molecular weight, and under the tradename Carbowax.

As used herein, the terms “raised surfaces or flat peaks” refer to the section of surface that are not the grooves, but are at an elevated position relative to the bottom of the grooves.

### DESCRIPTION OF THE FIGURES

The accompanying figures, which are incorporated into and form a part of the specification, illustrate several embodiments of the present invention and, together with the description, serve to explain the principles of the invention. The figures are only for the purpose of illustrating a preferred embodiment of the invention and are not to be construed as limiting the invention.

FIG. 1 shows a diagram illustrating both the one embodiment of the micro-patterned grooves A) with PLGA/drug deposited within the grooves and B) a micro-patterned surface of one device.

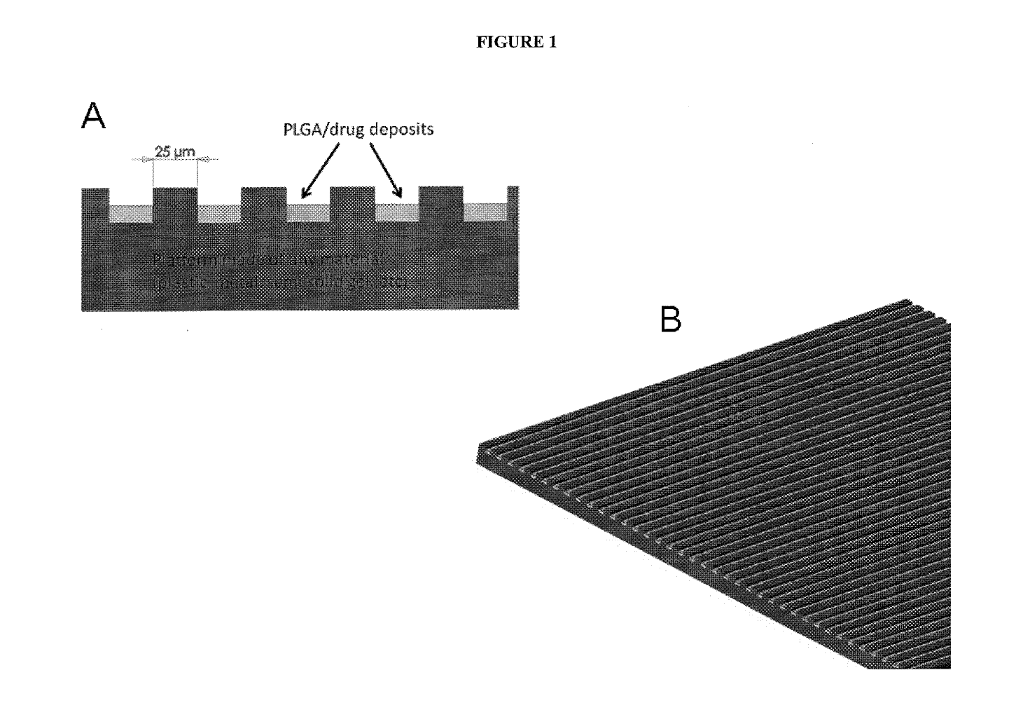

FIG. 2 shows another embodiment of the current invention wherein the micro-patterned grooves are 50 μm deep and 25 μm wide, the peaks are 25 μm wide, and the deposited therapeutic agent fills the grooves leaving 25 μm of the groove. The grooves are created at right angles to the peaks.

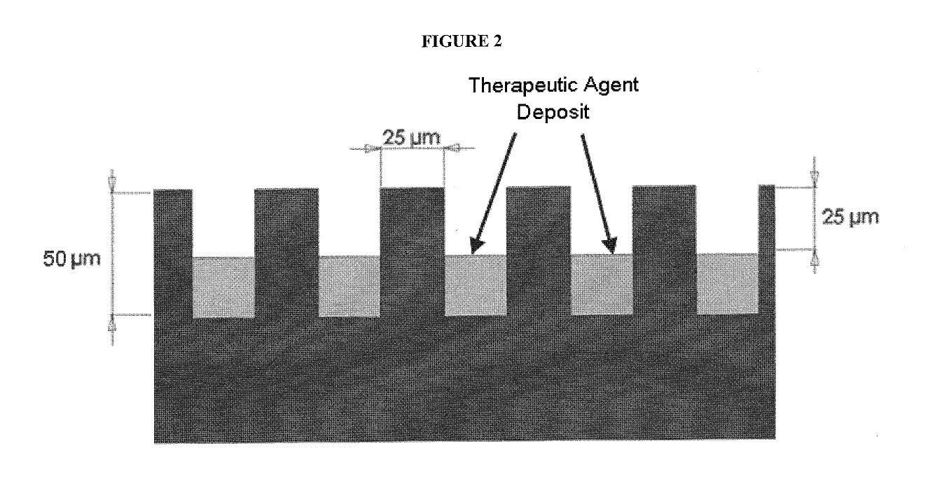

FIG. 3 shows the surface of one embodiment of the device (1)

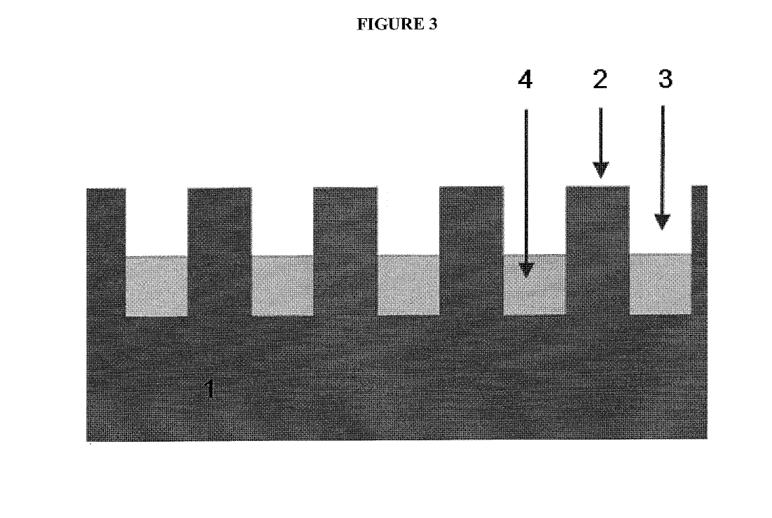

FIG. 4 shows a side cut view of a cylindrical embodiment of the device (1) with a central lumen (5) with the micro-patterned surface. The cylindrical embodiment of the device (1) and indicates the micro-patterned grooves containing medication (3) at the bottom of the grooves (4). The non-modified top surface (2) is at least 10-50 μm above the bottom of the grooves (4). This diagram also demonstrates the deposition of PLGA/drug within the grooves (4) of the surface modifications.

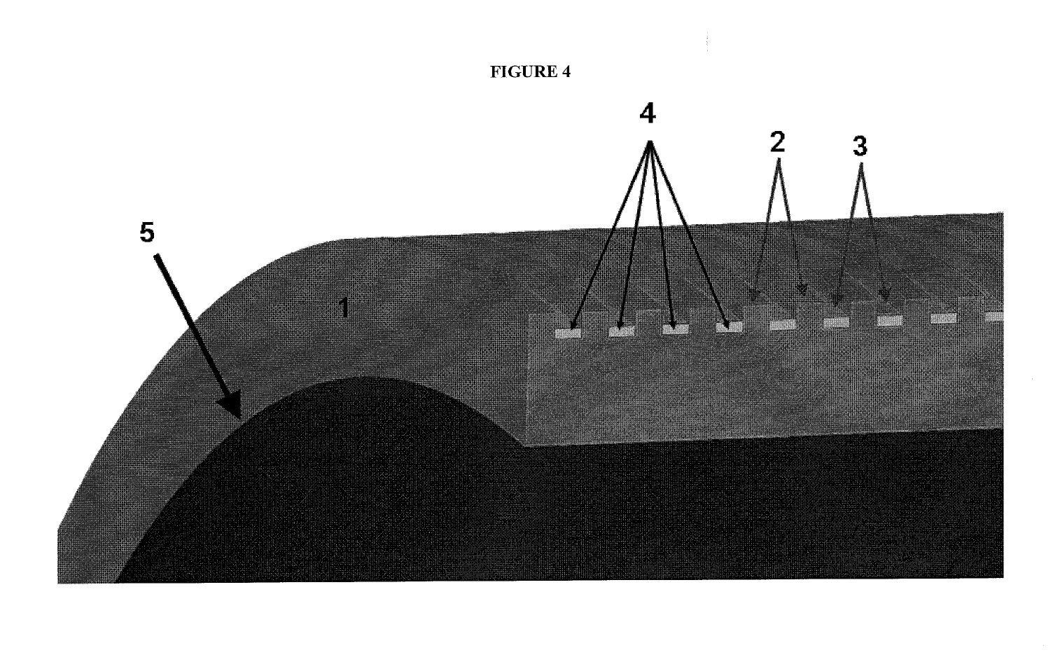

FIG. 5 shows another view of a cylindrical embodiment of the device (1) with a central lumen with the micro-patterned surface.

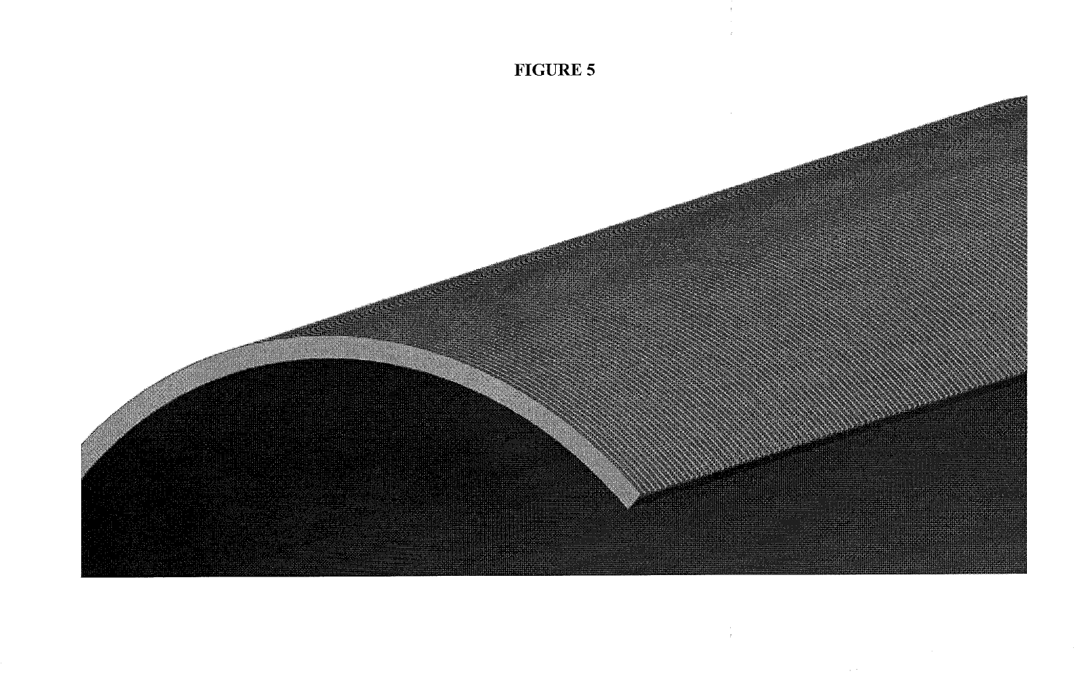

FIG. 6 shows a diagram of the one embodiment of a cylindrical device (1) with micro-patterned grooves (containing erodible material with medication) (3) on the surface of said device. This diagram was drawn with a cross-section taken out of the body of the device to illustrate the depth and geometry of the surface patterns. In this embodiment, the grooves are 25 μm wide and 25 μm deep and contain a 10 μm film of PLGA/drug within the grooves.

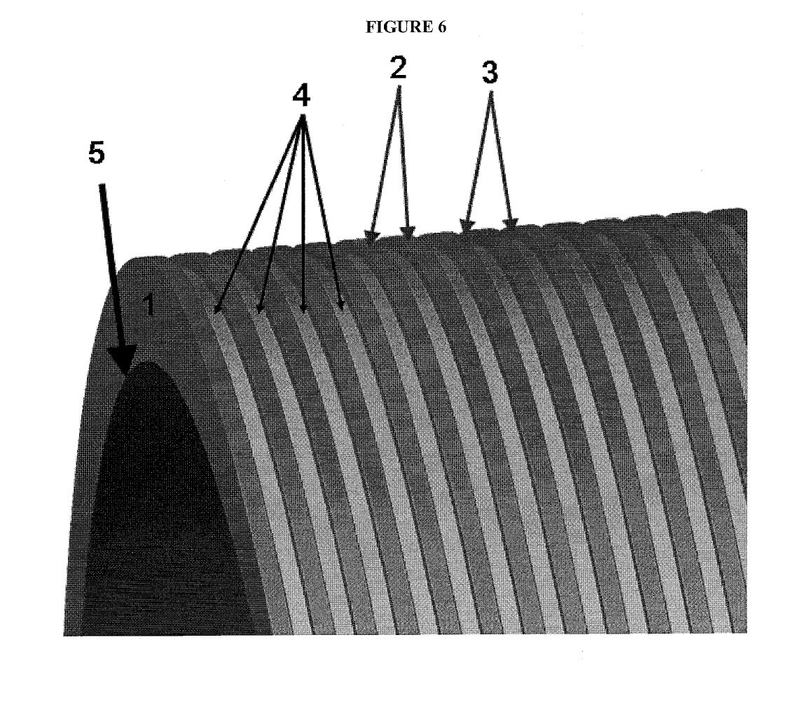

FIG. 7 shows a diagram of the one embodiment of a cylindrical device (1) with micro-patterned grooves (containing erodible material with medication) (3) on the surface of said device as well as micro-patterned grooves (containing erodible material with medication) (7) on the inner surface of the lumen of said device (5). The non-grooved top surface (2) and within the lumen (6) are at least 10-50 μm above the bottom of the grooves (4 and 8). This diagram was drawn with a cross-section removed from the body of thes device to illustrate the deposition of PLGA/drug within the grooves of the surface (4) and lumen (8).

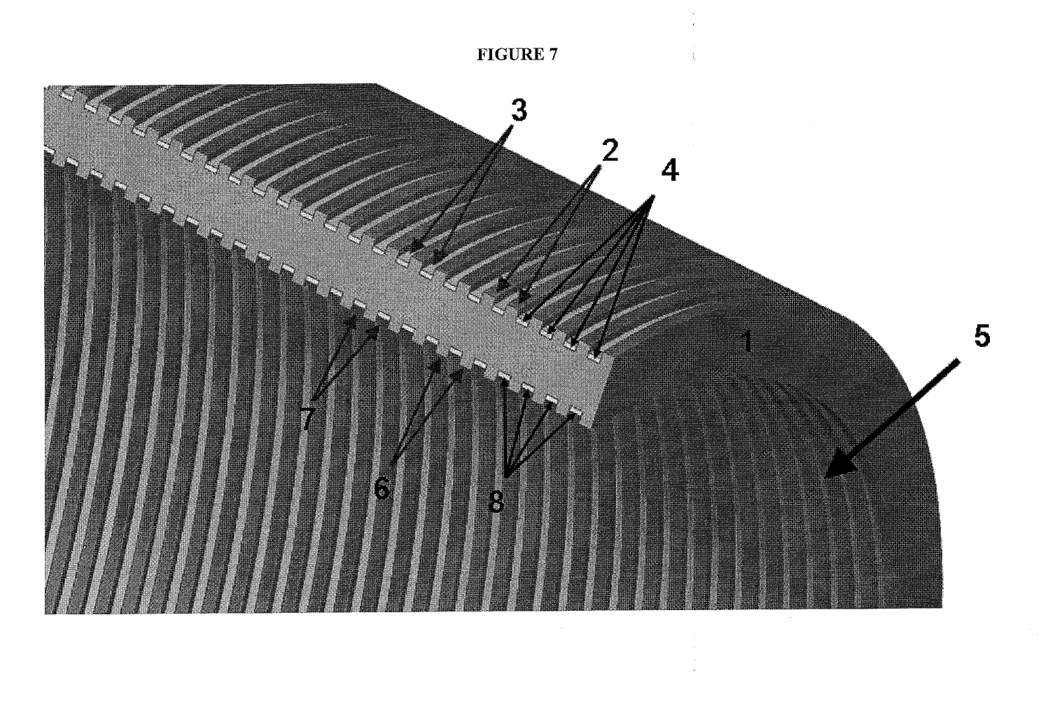

FIG. 8 shows use of triamcinolone acetonide (TA), a synthetic corticosteroid, in ocular tissue [8].

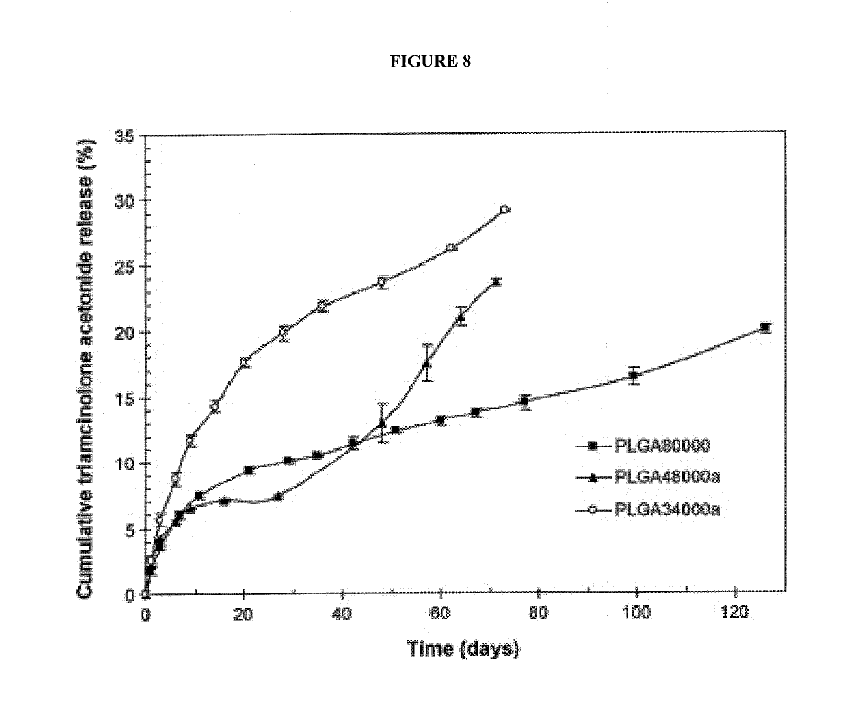

FIG. 9 shows the use of PLGA+rapamycin+BHT as a therapeutic agent in ocular tissue. BHT is an antioxidant and acts as a stabilizer to prevent oxidative degradation of rapamycin.

FIG. 10 shows a diagram of the manufacturing process.

FIG. 11 shows a diagram of the process workflow.

### LIST OF REFERENCE NUMERALS

1 the device
2 non-grooved top surface
3 micro-patterned grooves (containing erodible material with medication)
4 bottom of the micro-patterned grooves
5 the lumen of said device
6 non-grooved top surface within the lumen
7 micro-patterned grooves on the inner surface of the lumen
8 bottom of the micro-patterned grooves within the lumen

### DESCRIPTION OF THE PREFERRED EMBODIMENT

### 1. Background

One previous example of anti-fouling technology is found in Banerjee, I. et al. (2011) Adv. Mater. 23(6), 690-718 [9] incorporated herein by reference. This reference teaches several strategies for prevent fouling due to proteins, bacteria, and marine organisms. “Several design patterns, including channels, ridges, pillars, pits, and ribs (Sharklet AF, biomimetic topography inspired by shark skin), were fabricated on PDMS elastomer using standard photolithography techniques. Based on their studies of the performance of several microtopographies, they concluded that an effective coating should possess topographical features that are smaller than either the dimension of marine organisms or the parts of organisms that explore the surface while settling.” The reference does not contemplate a device comprising micro-patterned geometric pattern having anti-biofouling properties or material and do not combine a micro-patterned surface with a drug eluting material that controls both acute and chronic aspects of inflammation and cellular proliferation.

Another anti-fouling device is described in Ainslie, K. M. and Desai, T. A. (2008), Lab Chip 8(11), 1864-1878 [10] incorporated herein by reference. This review mentions that by adapting microfabrication techniques originally developed in the microelectronics industry novel device for drug delivery, tissue engineering and biosensing have been engineered for in vivo use and that implant microfabrication uses a broad range of techniques including photolithography, and micromachining to create devices with features ranging from 0.1 to hundreds of microns with high aspect ratios and precise features. With respect to biosensors, methods mentioned to prevent or limit capsule formation include anti-fouling polymers like PEG, biomimics such as phospholipids, flow based systems, membranes, and nanostructured surface topography, like nanowires. The reference does not disclose a device comprising an anti-fouling material having a micro-patterned geometric pattern and do not combine a micro-patterned surface with a drug eluting material that controls both acute and chronic aspects of inflammation and cellular proliferation.

Other antifouling materials are described by Vladkova, T. G. (2010) International Journal of Polymer Science 2010 (Article ID 296094), 22 pages [11] incorporated herein by reference. This reference discloses that many biocontact problems of polymer-based medical device may be solved using surface engineering that creates nanosize layers with controlled chemical composition, topography and roughness, and hydrophilic/hydrophobic balance. The reference teaches a variety of wafer coatings to prevent the adherence of cells and/or proteins following implantation. The reference also suggests that the effect of surface topography and chemistry on cellular response is of fundamental importance, especially where living systems encounter device surfaces in medical implants. Improved thrombo-resistance may be achieved by using: i) micro heterogeneous surfaces (e.g., polymers with micro phase separated structure and segmented polyurethanes); or ii) simulation of blood vessel properties (e.g., surfaces with hydrophilic nature and high mobility, negatively charged surfaces). For example, biomaterials with micro-domain surfaces allow adsorbed proteins to self-organize. Accordingly, surface microheterogeneity provides bioinert biomaterials. For example, low-trombogeneity of block co-polymers of the type ABA with a hydrophilic/hydrophobic micro-domain structure is due to a significant oppress of adhering platelets activation. Typical representative of this group are the segmented poly(etherurethanes). The reference does not contemplate a device comprising micro-patterned grooves having anti-biofouling properties and do not combine a micro-patterned surface with a drug eluting material that controls both acute and chronic aspects of inflammation and cellular proliferation.

Another anti-fouling strategy is described in Chen, S. et al. (2010) Polymer 51(23), 5283-5293 [12] incorporated herein by reference. This reference discloses that there are two major classes of biological anti-fouling materials, namely polyhydrophilic and polyzwitterionic materials. These materials are broadly grouped into PEG polymer-based materials, polybetaine materials, and polyampholyte materials. PEG anti-fouling materials have been well demonstrated to resist nonspecific protein adsorption and cell adhesion, but suffer from the disadvantage of biochemically-mediated oxidation. The reference teaches that hydrogen bonding and/or ionic interactions between these materials and the surrounding water molecules forms a hydration layer that is responsible for the anti-fouling properties. The reference does not disclose a device comprising an anti-fouling material having an etched geometric pattern and do not combine a micro-patterned surface with a drug eluting material that controls both acute and chronic aspects of inflammation and cellular proliferation.

Another anti-fouling strategy is described in Desai, T. A. et al. (2000) Biosens. Bioelectron. 15(9-10), 453-462 [13] incorporated herein by reference. This reference discloses the construction of implantable biosensors using anti-fouling materials. The reference describes several disadvantages of conventionally used anti-fouling coatings placed on biosensors that ultimately result in flaking, peeling, cracking and chipping. The reference discloses the construction of a nanopore biosensor chip comprising a plurality of filtration pores passing through conventional silicon wafers using conventional micro-patterning techniques. The reference teaches that micromachined membranes may be advantageous for in vitro and in vivo applications requiring membrane biostability and non-fouling over time. The data presented showed that little or no protein adhered to the silicon wafer nanopore membrane channels during the performance of a glucose diffusion test, whereas protein did adhere to ion-track etched (Millipore) or porous alumina (Whatman) compositions. The reference does not contemplate a device comprising micro-patterned grooves that provide a drug delivery platform and do not combine a micro-patterned surface with a drug eluting material that controls both acute and chronic aspects of inflammation and cellular proliferation.

Another anti-fouling strategy is described in Leoni, L. et al. (2002) Sensors 2(3), 111-120 [14] incorporated herein by reference. This reference discloses monodisperse nanoporous, biocompatible, silicon membranes as a platform for cell and/or drug delivery that remains free of fibrotic deposition following a two week implantation into a rat peritoneal cavity. Further, the wafers were compatible for in vitro growth of insulinoma and/or neurosecretory (PC12) cells that grew to confluence and differentiated within the nanoporous wells. The reference does not contemplate a device comprising micro-patterned grooves that provide a drug delivery platform and do not combine a micro-patterned surface with a drug eluting material that controls both acute and chronic aspects of inflammation and cellular proliferation.

Another anti-fouling strategy is described in Messersmith, P. B. et al. “Peptidomimetic Polymers for Antifouling Surfaces,” U.S. Pat. No. 7,618,937 [15] incorporated herein by reference. This reference discloses polymer-peptide composition that have anti-biofouling properties. These polymers include but not limited to polyethylene glycol (PEG), polyethylene oxide (PEO), polypropylene oxide (PPO), PEO-PPO-PEO block copolymers, polyphenylene oxid, PEG/tetraglyme, PMEMA, polyMPC, and perfluorinated-polyethers. The references suggests that it is the peptide portion of the composition that is responsible for the anti-fouling properties. The polymers are suggested for use as a coating to prevent protein and cellular adhesion to devices for medical and research applications. These devices may encompass medical implants, surgical devices, biological sample containers, diagnostic devices and/or biosensors. The reference does not contemplate a device comprising micro-patterned grooves having anti-biofouling properties and do not combine a micro-patterned surface with a drug eluting material that controls both acute and chronic aspects of inflammation and cellular proliferation.

Another anti-fouling strategy is described in Mirzadeh, H. et al. (1998) Iranian Polymer Journal 7(1), 5-13 [16] incorporated herein by reference. This reference describes the creation of super-hydrophobic polymer surfaces by laser treatment and turns them into hydrophilic ones grafting hexamethylacrylate (HEMA) after their preactivation by CO2-pulse laser treatment. The data from in vitro investigations demonstrate significantly reduced platelet adhesion and aggregation on the two type modified surfaces but the best regarding the blood compatibility appears to be the super-hydrophobic one. The reference does not contemplate a device comprising micro-patterned grooves having anti-biofouling properties and do not combine a micro-patterned surface with a drug eluting material that controls both acute and chronic aspects of inflammation and cellular proliferation.

Another anti-fouling strategy is described in Acikgoz, C. et al. (2011) Eur. Cell. Mater. 21(Suppl. 2), 39 [17] incorporated herein by reference. This reference describes a polymer, poly(2-methyl-2-oxazoline) (PMOXA), with an antibiotic moiety to kill bacteria adhering onto the surface. The reference does not contemplate a device comprising micro-patterned grooves having anti-biofouling properties and do not combine a micro-patterned surface with a drug eluting material that controls both acute and chronic aspects of inflammation and cellular proliferation.

Another anti-fouling strategy is described in Stofko Jr., J. J. and Yarwood, J. M. “Antimicrobial and Antifouling Polymeric Materials,” U.S. patent application Ser. No. 13/120,293 [18] incorporated herein by reference. The reference describes polymeric material that can be used, for example, to provide coatings that can be antifouling, antimicrobial, or both. The reference teaches that the polymeric material described has a plurality of different pendant groups that include a first pendant group containing a —COOH group or a salt thereof, a second pendant group containing a poly(alkylene oxide) group, a third pendant group containing a silicon-containing group, and a fourth pendant group containing a quaternary amino group. The reference does not contemplate a device comprising micro-patterned grooves or a geometric pattern having anti-biofouling properties and do not combine a micro-patterned surface with a drug eluting material that controls both acute and chronic aspects of inflammation and cellular proliferation.

One patent application, Nguyen et al. “Bare Metal Stent with Drug Eluting Reservoirs,” U.S. patent application Ser. No. 13/010,869 [19], incorporated herein by reference, describes therapeutic agents released under controlled and directional conditions from a stent. The reference does not contemplate a device comprising micro-patterned grooves or a geometric pattern having anti-biofouling properties and do not combine a micro-patterned surface with a drug eluting material that controls both acute and chronic aspects of inflammation and cellular proliferation.

Effects of a grooved surface on cell morphology are described by Chou, L. et al. (1995) J. Cell Set. 108(4), 1563-1573 [20] incorporated herein by reference. Human gingival fibroblasts were cultured on titanium coated grooved surfaces of 3 μm in depth. Cells on grooved surfaces were significantly elongated and orientated along the grooves of the substratum, while cell height, measured using confocal scanning laser microscopy, was ˜1.5-fold greater than that of cells on smooth surfaces. The surface modifications described here are on single micrometer scale and does not discuss the acute/chronic concept with etched/micro-patterned surfaces combined with medications.

Other effects of cells grown on nanopatterned surfaces, such as ‘nanopost’ and ‘nanograte’ structures, are described in Choi, C.-H. et al. (2007) Biomaterials 28(9), 1672-1679 [21] incorporated herein by reference. Human foreskin fibroblasts exhibited significantly smaller cell size and lower proliferation on needle-like nanoposts, and enhanced elongation with alignment on blade-like nanogrates. These phenomena became more pronounced as the nanotopographical three dimensionality (structural height) increased. The nanopost and nanograte architectures provided the distinct contact guidance for both filopodia extension and the formation of adhesion molecules complex, which was believed to lead to the unique cell behaviors observed. The surface modifications described here are on single nanomter scale and does not discuss the acute/chronic concept with etched/micro-patterned surfaces combined with medications.

Other effects of cells grown on nanopatterned surfaces, such as how nanotopology can affect cell adhesion and spreading, are described in Tay, C. Y. et al. (2011) Micro-/Nano-engineered Cellular Responses for Soft Tissue Engineering and Biomedical Applications, Small 7(10), 1361-1378 [22] incorporated herein by reference. The surface modifications described here are on single nanomter scale and does not discuss the acute/chronic concept with etched/micro-patterned surfaces combined with medications.

Other effects of cells grown on nanopatterned surfaces, such as how nanotopology can affect cell adhesion and spreading, are described in Bettinger, C. J., Langer, R., and Borenstein, J. T. (2009) Angewandte Chemie International Edition in English 48(30), 5406-5415 [23] incorporated herein by reference. Three basic nanotopography geometries include nanograting, nanopost array, and nanopit array are also described. The surface modifications described here are on single nanomter scale and does not discuss the acute/chronic concept with etched/micro-patterned surfaces combined with medications.

### 2. Description of the Invention

This invention is in the field of implantable medical devices. The present invention relates to a device constructed from metals, polymers or other materials that are amenable to precise surface modifications and coupling with erodible agents methods for its use, wherein (1) the erodible agents, which contain active ingredients (i.e., for example, medications) provide for acute control of cellular proliferation and (2) a pattered surface having micron-, and/or nano-sized micro-patterning characteristics that imparts anti-proliferative properties.

Further, the device comprises a drug delivery platform by placing erodible or non-erodable medication depots within the grooves of the constructed patterns. In other embodiments, device is created from a material wherein a pattered surface having micron-sized micro-patterned characteristics imparts anti-proliferative or anti-fibrotic properties. Further, the device comprises a drug delivery platform by placing medication depots (i.e., a plastic, or a semi-solid gel) within the grooves of the micro-patterned pattern.

For example, the device may have an etching pattern that forms a grid pattern or geometric pattern. Different devices can therefore be constructed with different grid dimensions or geometric patterns. The current invention contemplates that an implanted 10-50 μm, preferrably 20-35 μm, grid shows: i) a decrease in fibroblast or other cells number: and ii) an increase in cell alignment (i.e., improved organization of adhered cells). This is in comparison to a blank (non-micro-patterned or non-etched) device control that displays a disorganized pattern of more densely adhered cells. The current invention contemplates that the optimal dimension of the geometric patterns might depend on the specific material. Data, such as Table 1, shows that devices, made of various materials, having specific surface etching patterns can control fibroblast proliferation.

The invention further contemplates that medications may be placed in the grooves of the micro-patterned grid or geometric pattern such that the benefit of the micro-patterned surfaces preventing fibroblast growth and promoting organizations of the micro-patterned surfaces is maintained or supplemented/accentuated. The medications can include but are not limited to a steroid, rapamycin, everolimus, tacrolimus, paclitaxel or other antifibrotic medications as well as biologics or targeted therapeutics for specific diseases like glaucoma, macular degeneration or neurodegenerative diseases. Preferably, the medication would be placed in a slow release depot comprising a polymer including but not limited to PLGA, PLA, PGA or PCL.

Methods of the present invention are contemplated as implanting the devices within tissues for the treatment of various medical conditions without inducing fibrosis. For example, the medical condition may be inflammation and/or swelling wherein the implanted device facilitates drainage of a tissue. Once the device is placed, the depot slowly releases a medication (e.g., an antifibrotic) to prevent/lessen encapsulation of the device with fibroblasts or other cell types. Secondarily, once the depot has released all the medication, the micro-patterned surface of the device continues to inhibit the encapsulation process. Another method contemplated by the present invention is related to precisely depositing the medication depots within the geometric pattern grooves by using by precise means. In one embodiment, said medication depots are deposited by an inkjet printer or other precision dispensing instrument. The placement and amount of the medication depots are such that the antifibrotic properties of the micro-patterned grid or geometric pattern surface are maintained and contributes to an antifibrotic environment even before the complete release of the medication from the depot. Specifically, if the medication depot is deposited on the raised portions of the geometric pattern, the anti-fibrotic properties of the device are impaired.

### 3. Therapeutic Agents

Triamcinolone acetonide (TA), a synthetic corticosteroid, has been used to treat ocular tissue delivered using a number of controlled release systems such as PVA, PEVA/PBMA (SurModics), PLGA, PCL and PMM. The SurModics system claims up to 2 yrs of delivery whereas more typical durations of release are around 4-12 weeks.

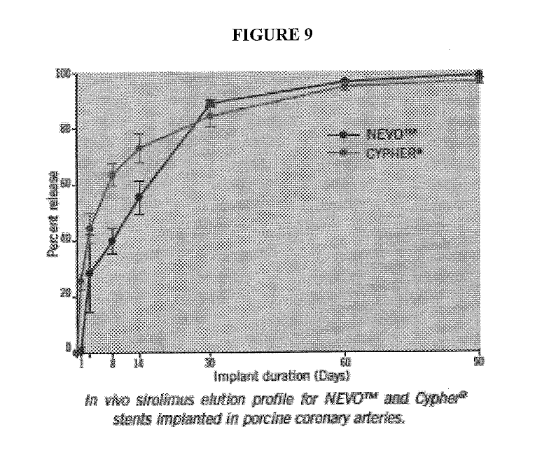

There is previous use of rapamycin, a synthetic corticosteroid, in ocular tissue using PLGA drug-eluting stents. One particular formulation was PLGA+rapamycin+butylated hydroxy toluene (BHT) as described in the Eurpean Patent Application EP2361593 [24]. BHT is an antioxidant and acts as a stabilizer to prevent oxidative degradation of rapamycin. Release from most thin film reservoir systems is somewhere in the 30-40 day range (see FIG. 9 from reference [25]; NEVO is a PLGA/rapamycin system). However, some studies suggest longer release rates are possible by switching to higher L:G ratios of PLGA (higher L:G ratio means more hydrophobic polymer; as a result, less water can swell into the system so diffusion of the drug out of the polymer is slower; however, higher L:G ratios also mean slower biodegradation of the PLGA).

Bevacizumab has been conjugated with PEG and encapsulated in PLGA nanoparticles Pan et al. (2011) [26]. Conjugation is an actual chemical bonding of the polymer to the drug where encapsulation is just a physical barrier so these represented very different delivery strategies. Neither system showed very favorable release control. Another study, formulated PLGA nanoparticles with bevacizumab and achieved 4 weeks of sustained delivery, Boddu et al. (2010) [27]. Stability of the protein is of concern since as PLGA degrades its byproducts are lactic acid and glycolic acid so the PLGA matrix can become an acidic environment.

### 4. Use of the Device

In one embodiment, the present invention contemplates a drug delivery device wherein medication (i.e., for example, antifibrotics and other medications or therapeutic agents) is placed within a plurality of grooves such that the medication does not rise above the top surface of the grooves. Although it is not necessary to understand the mechanism of an invention, it is believed that this medication placement maintains the benefit of the micro-patterned surfaces for preventing fibroblast growth and/or promoting organizations. It is also believed that such medication placement inhibits organized cell proliferation along the micro-patterned geometric grooves so that encapsulation and scar tissue formation is minimized, eliminated, or appreciably reduced. Further, a distinct reduction in cell proliferation may result. In one embodiment, medication can be a steroid, rapamycin, or other antifibrotic medications as well as biologics or targeted therapeutics for specific diseases including, but not limited to cataract, diabetic manifestations in the eye, systemic disease manifestations in the eye, inherited retina and choroidal diseases, glaucoma, neuropathies/neurodegenerative disease, uveitic diseases, or macular degeneration (wet and dry). In one embodiment, the medication may be placed in a slow release depot such as PLGA or PEG systems (or other). The devices can then be implanted inside of tissues and benefit from the action of both the depot/medication and the micro-patterned surfaces. Once the device is placed, primarily, the depot/medication will slowly release anti-fibrotic or other medications to prevent/lessen encapsulation of the device with fibroblasts or other cell types. This might benefit the device action, which could be for drainage or for other purposes unique to any given implant. Secondarily, once the medication and the depot have diffused, the micro-patterned surface remains so that it will still lessen the encapsulation process independent of the depot medication. The medication depot may be placed in such a way (with precise inkjet deposition, for example) that may allow the micro-patterned surface qualities to be maintained and provide an antifibrotic environment even before the medication/depot empties from the bottom of the grooved space.

In one embodiment, PLGA matrices release drug over immediate period after implantation, preventing initial cell proliferation response, (other depot mechanisms other than PLGA might also be used). In another embodiment, micro-patterned surface may provide initial and long-term inhibition of fibrosis, ensuring long-term prevention of capsulation. In one embodiment, precise inkjet printing may provide the avenue of deposition for the medication in the bottom of the micro-patterned grooved space. Inkjet printing is able to accurately fill such micron-scale features with flexibility in the solution to be dispensed and high throughput capability. In another embodiment, PLGA/drug matrix must be contained within “channels” of surface as a conformal polymer coating may counteract beneficial effect of micro-patterned surface.

### 5. Detailed Description of the Invention

The following detailed description, and the drawings to which it refers, are provided for the purpose of describing and illustrating certain preferred embodiments or examples of the invention only, and no attempt has been made to exhaustively describe all possible embodiments or examples of the invention. Thus, the following detailed description and the accompanying drawings shall not be construed to limit, in any way, the scope of the claims recited in this patent application and any patent(s) issuing there from.

Distinguishing features compared to other devices:
1. No mechanically moving parts
2. Micro-patterned grooved valleys (i.e., for example, approximately 10-50 μm wide and at least 25 μm deep)
3. Flat peaks (i.e., for example, at least 10-50 μm wide)
4. The angle between the peaks and the valleys are at a right angle (e.g., 90 degrees) in some materials but curved in other materials (i.e., for example, between approximately 95 to 120 degrees, as a frame of reference 180 degrees would be a line crossing all the peaks). The curve is the slope between the peak and the wall going down to the bottom of the groove (like a mountain rather than a cliff).
5. The dimensional ratios of peaks and valleys and angles of the surface modifications are specific to each given material used as it relates to the interaction of material to dimensions to proliferating cells
6. The coupling of micro-patterned surfaces with eluting medication depots is at the core of the present invention and addresses both acute and chronic aspects of the biologic response to implanted materials.

In one embodiment, the invention contemplates implanted medical device in or on the eye. In one embodiment, the invention contemplates implanted medical device in the sclera. In one embodiment, the invention contemplates implanted medical device in Schlemm's canal. The present invention considers a device made by various materials. In one embodiment, the material is polymeric such as silicone, polyimide (PI), polysulfone (PES), polyetheretherketone (PEEK), polyetherimide (PEI), or metallic materials such as titanium or aluminum or ceramic such as titanium oxide, calcium phosphate or hydroxyapatite or alloys such as nickel-titanium (NiTi), stainless steel or titanium alloys.

In one embodiment, the present invention contemplates an implanted medical device capable of having a variety of shapes. In one embodiment, the device has shape selected from the group consisting of spherical, non-spherical (egg-shaped), cylindrical, rectangular, cubic, toroidal, conical, cuboidal, pyramidal, prism, and planar shapes. In one embodiment, the device has a cylindrical shape. In one embodiment, the device has a cylindrical shape contains at least one lumen. In one embodiment, the lumen contains a depot. In one embodiment, said depot contains at least one medication. In one embodiment, said medication includes, but is not limited to, anti-fibrotic agent, anti-inflammatory agent, immunosuppressant agent, anti-neoplastic agent, migration inhibitors, anti-proliferative agent, rapamycin, triamcinolone acetonide, everolimus, tacrolimus, paclitaxel, actinomycin, azathioprine, dexamethasone, cyclosporine, bevacizumab, an anti-VEGF agent, an anti-IL-1 agent, canakinumab, an anti-IL-2 agent, viral vectors, beta blockers, alpha agonists, muscarinic agents, steroids, antibiotics, non-steroidal anti-inflammatory agents, prostaglandin analogues, ROCK inhibitors, nitric oxide, endothelin, matrixmetalloproteinase inhibitors, CNP/BMP, corticosteroids, and/or antibody-based immunosuppresants. In one embodiment, said medication is combined with a polymer. In one embodiment, said polymer is selected from the group consisting of poly(lactic-co-glycolic acid), polyethylene glycol, poly(lactic acid), poly(glycolic acid), poly(amido ester), polyethylene terephthalate, poly(caprolactone), poly(hydroxy butyrate), poly(butylene succinate), poly(vinyl alcohol), poly(hydroxybutyrate), poly(methyl acrylate), poly(methyl methylmethacrylate), poly(sebacic acid), carboxymethyl cellulose, ethyl cellulose, cellulose acetate, polydioxanone, or polymers from the categories: polyesters, polyanhydrides, polyamides, polycyanoacrylates, polyurethanes, polyorthoesters, silicones, acrylic polymers, cellulose derivatives or poloxamers.

It is not intended that the current device be limited to one particular shape or be limited to any particular geometrical pattern. In one embodiment, said device has shape selected from the group consisting of spherical, non-spherical (egg-shaped), cylindrical, rectangular, cubic, toroidal, conical, cuboidal, pyramidal, prism, and planar shapes. In one embodiment, said device has micro-patterned grooves in a grid pattern. In one embodiment, said device has micro-patterned grooves in a vertical orientation. In one embodiment, said device has micro-patterned grooves in a horizontal orientation. In one embodiment, said device has micro-patterned grooves in a diagonal orientation. In one embodiment, said micro-patterned grooves intersect. In the case of a spherical shape, the micro-patterned grooves could be in the form of circular pattern about the body of the device. In the case of a toroidal shape, the micro-patterned grooves could be in the form of circular pattern about the body of the device. In the case of cylindrical, rectangular, cubic, cuboidal, and planar shapes, the micro-patterned grooves could be in the form of vertical, horizontal, diagonal, or intersecting grids. In the case of c conical, pyramidal, prism shapes, the micro-patterned grooves could be in the form of vertical, horizontal, diagonal, intersecting grids in the form of circular pattern about the body of the device. In one embodiment, the device with a cylindrical shape has micro-patterned grooves in a vertical orientation. In one embodiment, the device with a cylindrical shape has micro-patterned grooves in a horizontal orientation. In one embodiment, the device with a cylindrical shape has micro-patterned grooves in a diagonal orientation.

In one embodiment, said device is a catheter. In one embodiment, said device is a stent. In one embodiment, said device is a catheter for a defibrillation device. In one embodiment, said device is a catheter for a defibrillation device. In one embodiment, said device is a intravenous catheter. In one embodiment, said device is a Hickman line. In one embodiment, said device is a mesh prosthesis. In one embodiment, said device is a hernia mesh. In one embodiment, said device is a Baerveldt glaucoma implant. In one embodiment, said device is a glaucoma shunting device. Baerveldt glaucoma implants feature a surface area plate. In one embodiment, the current invention contemplates micro-patterning of the plate of the Baerveldt device to prevent encapsulation.

In one embodiment, the use of the implant prevents disorderly growth of fibroblasts. In one embodiment, the use of the implant prevents the formation of scar tissue. In one embodiment, the micro-patterned grooves provide an avenue for drainage.

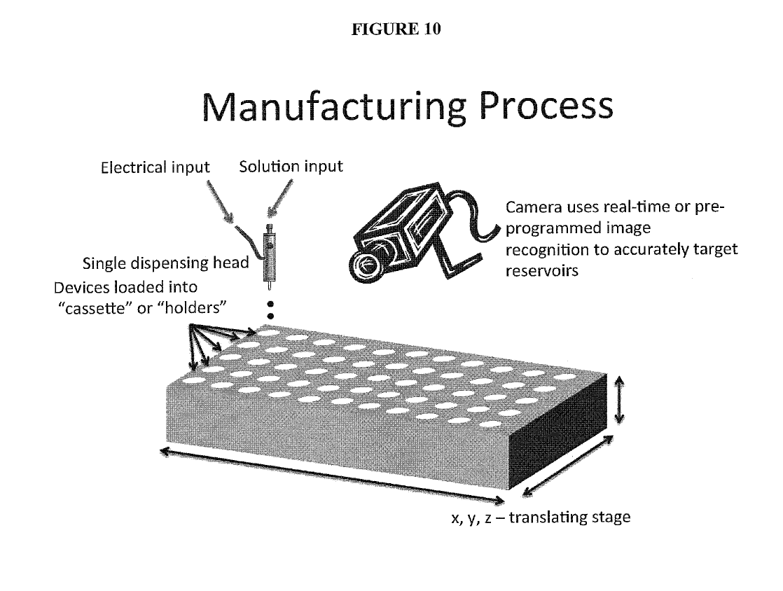

In one embodiment, the current invention uses an inkjet loading system to deposit therapeutic agents into the micro-patterned grooves. The drop volumes produced with the inkjet dispensing system are in the range of 1.5 pL to 4.2 nL. The system provides precise control of filling volumes, typically 1-3% repeatability (drop-to-drop, depending on dispensing solution properties), with a drop firing rate up to about 30,000 per second. Such a system has high throughput, simple operation, high versatility, and is relatively inexpensive. Error! Reference source not found. shows stent loading with an injection loading system. The entire stent could be loaded in a very rapid and precise process. The system is largely automated with machine vision-based mapping of deposition locations and accurately ejected drops to those locations appropriately, as illustrated in FIG. 10. FIG. 10 shows a diagram of the manufacturing process. The system employs a real-time camera or pre-programmed image recognition to accurately target reservoirs/depots.

In one embodiment, a solution containing approximately 10% 75:25 lactide:glycolide PLGA, 10% rapamycin and 0.5% BHT in DMSO are prepared as the dispensing solution. This solution is loaded into the inkjet dispenser, which is heated to 50° C. to facilitate the dispensing process. The control software uses the reservoir location map to translate the devices underneath the inkjet dispenser such that the dispensing locations pass under the inkjet tip. Translation is accomplished by three-axis motion stages and controllers connected to a computer via a hardware interface. The inkjet dispenser is triggered in a “drop-on-demand” mode by software control to dispense a set number of droplets of the solution into the grooves, filling the grooves completely but not overflowing them so as to prevent deposition of the matrix onto the raised surfaces. After dispesing the solution into the grooves in multiple devices in a batch process, the devices are transferred to a vacuum oven and the DMSO is driven off leaving only PLGA, rapamycin and BHT. This process is repeated, successively filling and drying the reservoirs until the solids comprise the desired depot dimensions. This process workflow is shown diagrammatically in Error! Reference source not found.

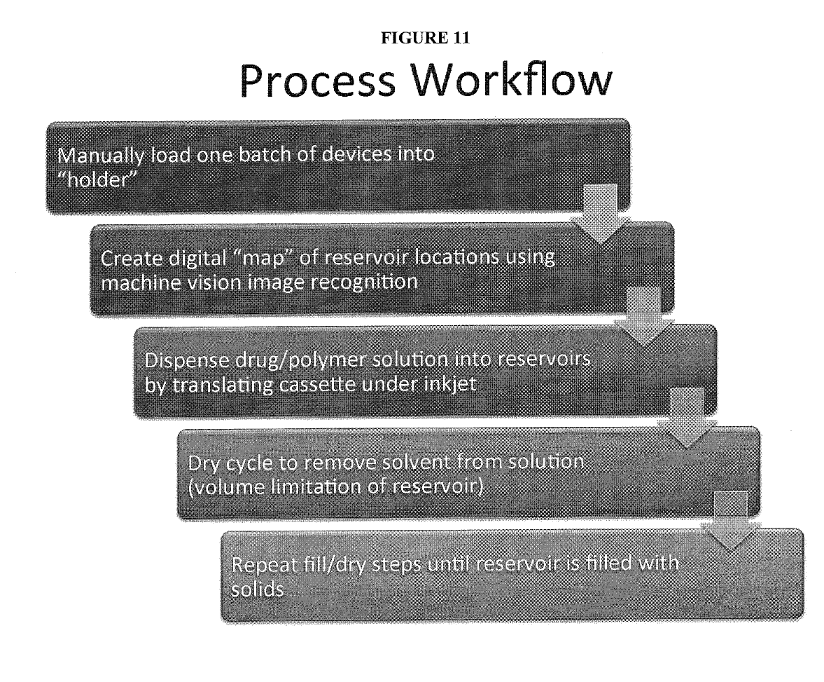

FIG. 11 shows a diagram of the process workflow. Initially one manually loads one batch of devices into “holder.” Secondly, one creates digital “map” of reservoir locations using machine vision image recognition. Subsequently, one dispenses therapeutic agent or drug/polymer solution into reservoirs by translating cassette under inkjet. Followed by a dry cycle to remove solvent from solution (volume limitation of reservoir), then the process is repeated with fill/dry steps until reservoir is filled with solids

### 6. Supporting Data

TABLE 1
Cell growth associated with micro-patterned surfaces in various materials. Each
data point was compared to a non micro-patterned surface as the control (data not shown). All
non micro-patterned surfaces resulted in diffuse growth of cells. The patterns were created with
equivalent width and depth.

| Material | 0.5 μm | 1.0 μm | 5.0 μm | 10.0 μm | 20.0 μm | 25.0 μm | 30.05 μm | 50.0 μm | 100.0 μm |
|---|---|---|---|---|---|---|---|---|---|
| Silicone | D | D | D | D | Mo | Mo | Mi | D | D |
| PEEK | D | D | D | Mo | Mi | Mi | Mi | D | D |
| Titanium | D | D | D | Mo | Mi | Mi | Mi | Mo | D |
| Stainless Steel | D | D | D | D | Mo | Mo | Mi | Mo | D |
| PTFE | D | D | D | D | Mo | Mi | Mi | Mi | D |

D = Diffuse Cell Growth
Mo = Moderate Cell Growth
Mi = Minimal Cell Growth
PEEK = Polyether ether ketone
PTFE = Polytetrafluoroethylene

Thus, specific compositions and configurations of antiproliferative surface modifications and methods of use have been disclosed. It should be apparent, however, to those skilled in the art that many more modifications besides those already described are possible without departing from the inventive concepts herein. The inventive subject matter, therefore, is not to be restricted except in the spirit of the disclosure. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

### REFERENCES

1. Kahook, M. Y. “Antiproliferative surface modifications and methods of use,” U.S. Provisional Patent Application 61/640,843, filed May 1, 2012. (published N/A).
2. Freemantle, N. et al. (1999) “beta Blockade after myocardial infarction: systematic review and meta regression analysis,” BMJ 3/8(7200), 1730-1737.
3. Cruickshank, J. M. (2010) “β blockers in hypertension,” The Lancet 376(9739), 415.
4. Fleisher, D., Bong, R., and Stewart, B. H. (1996) “Improved oral drug delivery: solubility limitations overcome by the use of prodrugs,” Adv. Drug Delivery Rev. 19(2), 115-130.
5. Smith, C. D. et al. (1994) “A sensitive assay for taxol and other microtubule-stabilizing agents,” Cancer Lett. 79(2), 213-219.
6. Mooberry, S. L., Stratman, K., and Moore, R. E. (1995) “Tubercidin stabilizes microtubules against vinblastine-induced depolymerization, a taxol-like effect,” Cancer Lett. 96(2), 261-266.
7. Ro, A. J., Falotico, R., and Davé, V. (2012) “Morphological and degradation studies of sirolimus-containing poly(lactide-co-glycolide) discs,” Journal of Biomedical Materials Research Part B: Applied Biomaterials 100B(3), 767-777.
8. Eperon, S. et al. (2008) “A biodegradable drug delivery system for the treatment of postoperative inflammation,” Int. J. Pharm. 352(1-2), 240-247.
9. Banerjee, I., Pangule, R. C., and Kane, R. S. (2011) “Antifouling Coatings: Recent Developments in the Design of Surfaces That Prevent Fouling by Proteins, Bacteria, and Marine Organisms,” Advanced Materials 23(6), 690-718.
10. Ainslie, K. M. and Desai, T. A. (2008) “Microfabricated Implants for Applications in Therapeutic Delivery, Tissue Engineering, and Biosensing,” Lab Chip 8(11), 1864-1878.
11. Vladkova, T. G. (2010) “Surface Engineered Polymeric Biomaterials with Improved Biocontact Properties,” International Journal of Polymer Science 2010 (Article ID 296094), 22 pages.
12. Chen, S. et al. (2010) “Surface hydration: Principles and applications toward low-fouling/nonfouling biomaterials,” Polymer 51(23), 5283-5293.
13. Desai, T. A. et al. (2000) “Nanoporous anti-fouling silicon membranes for biosensor applications,” Biosens. Bioelectron. 15(9-10), 453-462.
14. Leoni, L., Attiah, D., and Desai, T. (2002) “Nanoporous Platforms for Cellular Sensing and Delivery,” Sensors 2(3), 111-120.
15. Messersmith, P. B. et al. “Peptidomimetic polymers for antifouling surfaces,” U.S. Pat. No. 7,618,937, application Ser. No. 11/280,107, filed Nov. 16, 2005. (issued Nov. 17, 2009).
16. Mirzadeh, H., Khorasani, M., and Sammes, P. G. (1998) “Laser surface modification of polymers. A novel technique for the preparation of blood compatible materials (II): in vitro assay,” Iranian Polymer Journal 7(1), 5-13.
17. Acikgoz, C. et al. (2011) “Implant Infection (Antifouling and Antimicrobial Surface Coatings through Poly(2-methyl-2-oxazoline)),” Eur. Cell. Mater. 21(Suppl. 2), 39.
18. Stofko Jr., J. J. and Yarwood, J. M. “Antimicrobial and Antifouling Polymeric Materials,” United States Patent Application Publication Number 20110171158, application Ser. No. 13/120,293, filed Aug. 25, 2009. (published Jul. 14, 2011).
19. Nguyen, T. M., Parker, T. L., and Shanley, J. F. “Bare Metal Stent with Drug Eluting Reservoirs,” United States Patent Application Publication Number 20110137407, application Ser. No. 13/010,869, filed Jan. 21, 2011. (published Jun. 9, 2011).
20. Chou, L. et al. (1995) “Substratum surface topography alters cell shape and regulates fibronectin mRNA level, mRNA stability, secretion and assembly in human fibroblasts,” J. Cell Sci. 108(4), 1563-1573.
21. Choi, C.-H. et al. (2007) “Cell interaction with three-dimensional sharp-tip nanotopography,” Biomaterials 28(9), 1672-1679.
22. Tay, C. Y. et al. (2011) “Micro-/Nano-engineered Cellular Responses for Soft Tissue Engineering and Biomedical Applications,” Small 7(10), 1361-1378.
23. Bettinger, C. J., Langer, R., and Borenstein, J. T. (2009) “Engineering substrate topography at the micro- and nanoscale to control cell function,” Angewandte Chemie International Edition in English 48(30), 5406-5415.
24. Nguyen, T. M., Parker, T. L., and Shanley, J. F. “Bare metal stent with drug eluting reservoirs,” European Patent Application EP 2361593, Application 11152966.5, filed Feb. 1, 2011. (published Aug. 31, 2011).
25. Falotico, R. et al. (2009) “NEVO™: a new generation of sirolimus-eluting coronary stent,” EuroIntervention 5(Supplement F), F88-F93.
26. Pan, C. K. et al. (2011) “Comparison of Long-Acting Bevacizumab Formulations in the Treatment of Choroidal Neovascularization in a Rat Model,” J. Ocul. Pharmacol. Ther 27(3), 219-224.
27. Boddu, S. H. S. et al. (2010) “Novel Nanoparticulate Gel Formulations of Steroids for the Treatment of Macular Edema,” J. Ocul. Pharmacol. Ther 26(1), 37-48.

1. A device comprising an anti-proliferative surface, wherein said surface comprises a micro-patterned geometrical pattern, said pattern having a plurality of grooves between a plurality of raised surfaces.

2. The device of claim 1, wherein said pattern is selected from the group consisting of vertical, horizontal, parallel vertical, parallel horizontal, circular, intersecting grid, and concentric rings.

3. The device of claim 1, wherein said grooves comprise a plurality of medication depots such that the top of said depots are below said plurality of raised surfaces.

4. The device of claim 1, wherein said plurality of raised surfaces are separated by a distance of approximately 10-50 μm.

5. The device of claim 1, wherein said plurality of raised surfaces are each less than 50 μm in width.

6. (canceled)

7. The device of claim 1, wherein said grooves are at least as deep as the distance separating said plurality of raised surfaces.

8. (canceled)

9. The device of claim 3, wherein said medication depots are at least 25 μm below said raised surfaces.

10. (canceled)

11. The device of claim 1, wherein said geometrical pattern inhibits cellular proliferation, cell attachment, cell migration or release of specific factors.

12. The device of claim 1, wherein said device is implanted in a tissue selected from the group consisting of an ocular tissue and an intraocular tissue.

13-14. (canceled)

15. The device of claim 1, wherein said surface further comprises silicone, polyimide (PI), polysulfone (PES), polyetheretherketone (PEEK), polypropylene, polyetherimide (PEI), titanium, nitinol, stainless steel, gold, hydrophilic or hydrophopic polymers, shape memory polymers or alloys, ceramics, alloys, silicates, or other materials.

16-17. (canceled)

18. The device of claim 1, wherein said device contains at least one lumen.

19. The device of claim 18, wherein said lumen contains said medication depot comprising at least one medication.

20. The device of claim 3, wherein said medication depot comprises at least one medication.

21. The device of claim 20, wherein said at least one medication is selected from the group consisting of anti-fibrotic agent, anti-inflammatory agent, immunosuppressant agent, anti-neoplastic agent, migration inhibitors, anti-proliferative agent, rapamycin, triamcinolone acetonide, everolimus, tacrolimus, paclitaxel, actinomycin, azathioprine, dexamethasone, cyclosporine, bevacizumab, an anti-VEGF agent, an anti-IL-1 agent, canakinumab, an anti-IL-2 agent, viral vectors, beta blockers, alpha agonists, muscarinic agents, steroids, antibiotics, non-steroidal anti-inflammatory agents, prostaglandin analogues, ROCK inhibitors, nitric oxide, endothelin, matrixmetalloproteinase inhibitors, CNPA, corticosteroids, and antibody-based immunosuppresants.

22. (canceled)

23. The device of claim 20, wherein said at least one medication is combined with a polymer selected from the group consisting of poly(lactic-co-glycolic acid), polyethylene glycol, poly(lactic acid), poly(glycolic acid), poly(amido ester), polyethylene terephthalate, poly(caprolactone), poly(hydroxy butyrate), poly(butylene succinate), poly(vinyl alcohol), poly(hydroxybutyrate), poly(methyl acrylate), poly(methyl methylmethacrylate), poly(sebacic acid), carboxymethyl cellulose, ethyl cellulose, cellulose acetate, polydioxanone, or polymers from the categories: polyesters, polyanhydrides, polyamides, polycyanoacrylates, polyurethanes, polyorthoesters, silicones, acrylic polymers, cellulose derivatives or poloxamers.

24-28. (canceled)

29. The device of claim 1, wherein said device is selected from the group consisting of a catheter, a stent, a defibrillation device, an intravenous catheter, a Hickman catheter, a mesh prosthesis, a hernia mesh, a Baerveldt glaucoma implant, a dental implant and a glaucoma shunting device.

30-124. (canceled)
